(19) United States
(12) Patent Application Publication
(10) Pub. No.: US 2009/0092715 A1
(43) Pub. Date: Apr. 9, 2009

(54) System for real-time characterization of ruminant feed rations

(76) Inventor: James F. Beck, Rogers, MN (US)

Correspondence Address: Moss & Barnett P.A., 4800 Wells Fargo Center, 90 South Seventh Street, Minneapolis, MN, 55402-4129, US

(21) Appl. No.: 12/151,194

(22) Filed: May 5, 2008

Related U.S. Application Data
(63) Continuation-in-part of application No. 11/881,481, filed on Jul. 27, 2007.
Application No. 11/881,481 is a continuation-in-part of application No. 11/494,312, filed on Jul. 27, 2006.
Application No. 11/494,312 is a continuation-in-part of application No. 11/881,490, filed on Jul. 27, 2007.
Application No. 11/881,490 is a continuation-in-part of application No. 11/494,312, filed on Jul. 27, 2006.

(51) Int. Cl. G01N 33/02; G01N 21/84; A23K 1/18

(52) U.S. Cl. 426/231

(57) ABSTRACT

A computer-based system for characterizing in real time the nutritional components of one of more ingredients for a ruminant feed ration, including dry matter, NDF, NDFd, lignified NDF ratio, peNDF, percent starch, IVSD, and particle size for a forage material; and IVSD and particle size for a grain material. The system utilizes proprietary NIRS equations based upon prior samplings of a variety of crop species like dual-purpose corn silage, leafy corn silage, brown midrib (“BMR”) corn silage, grass (silage/dry), alfalfa (silage/dry), BMR forage sorghum, normal dent starch grain, floury endosperm starch grain, vitreous endosperm grain, and steam-flaked corn grain, and applies those equations to current samplings of a corresponding crop to predict in real time the characteristics of such forage or grain material. The real-time characterization system may also utilize the predicted data to calculate a “total ration fermentation index” value that takes into account the total NDFd and IVSD characteristics (including RAS and RBS) of the forage and starch ingredients to be used in a feed ration to ensure that the ration will not contribute too much or too little digestibility to the cow. A “flash fermentation index” identifies feed formulations, based upon the real-time characterizations of the ingredients, that are too “hot” to feed to the cows without incurring the risk of lost production and adverse health issues.

### CROSS-REFERENCE TO RELATED APPLICATION

This application is a continuation-in-part of U.S. Ser. No. 11/881,481 filed on Jul. 27, 2007, which is a continuation-in-part of U.S. Ser. No. 11/494,312 filed on Jul. 27, 2006; and is a continuation-in-part of U.S. Ser. No. 11/881,490 filed on Jul. 27, 2007, which is a continuation-in-part of U.S. Ser. No. 11/494,312 filed on Jul. 27, 2006; all of which are hereby incorporated by reference in their entirety.

### FIELD OF THE INVENTION

The present invention relates to a system for screening a crop plant for the plant's starch digestion, fiber digestion and/or nutritional composition characteristics. More particularly, the present invention is a system for accurately predicting the starch and fiber digestion characteristics of a crop plant by Near Infrared Spectrometer (“NIRS”) analysis while preserving the identity of the crop plants, and connecting the predicted data to rumen function to accurately simulate the fermentability of a feed ration in a rumen animal's stomach to enable the creation of feed formulations that result in optimum productivity of ruminant animals.

### BACKGROUND OF THE INVENTION

Feed ration costs account for 45-60% of the total cost of producing milk, so optimal nutrition is important. Ideally, appropriate nutrient levels should be maintained, while feed costs are carefully maintained. Such optimal nutrition will enhance milk production, improve overall health of the cow, and reduce associated costs like veterinary bills, drug treatments, and breeding.

The main nutrient categories of importance for dairy cow rations are carbohydrates, fats, proteins, minerals, vitamins, and water. While fiber is not strictly a nutrient by definition, it critically affects the cow's digestion, and therefore must be considered by the dairy farmer or nutritionist when formulating feed rations. The undigested feed and digesta from the reticulum pass to the rumen, which essentially acts as a large fermentation vat. Holding 40 to 60 gallons of material, it also contains an estimated 150 billion bacteria, protozoa, and fungi per teaspoon of content. If fed a proper balance of forages and grain, the resulting 5.8-6.4 pH and 100-108° F. conditions within the rumen should allow the growth of these important microorganisms.

Through a process of rumination, the cow reduces the particle size of feed in the rumen, which enhances microbial function, and allows for easier passage out of the stomach compartments. Due to its strong musculature, the rumen allows mixing and churning of the digesta.

The objective of feeding dairy cows nutritionally balanced diets is to provide a rumen environment that maximizes microbial production and growth. The microbial population within the rumen consists of bacteria, protozoa, and fungi. Rumen pH is one of the most variable factors which can influence this microbial population and the levels of volatile fatty acids produced. The fiber digesters are most active at pH=6.2-6.8. Cellulolytic bacteria and methanogenic bacteria can be reduced when the pH begins to fall below 6.0. The starch digester microbes prefer a more acidic environment with a pH=5.2-6.0. Certain species of protozoa can be greatly depressed with a pH below 5.5. To accommodate all of these needs, normal feeding practices should be maintained at pH=5.8-6.4.

Within the rumen, these microorganisms can digest carbohydrates, proteins, and fiber. Through this digestion process, volatile fatty acids (“VFA”) and microbial protein that can be utilized by the animal are produced. Both structural (NDF) and non-structural (sugar and starches) carbohydrates undergo microbial fermentation in the rumen to produce VFA like acetic, propionic, butyric, isobutyric, valeric and isovaleric acids, and traces of various other acids. Acetic acid can constitute 50-60% of the total VFA and predominate in a high-forage diet. Production of adequate levels of acetate in the rumen is essential to maintain adequate levels of milk fat. Meanwhile, propionic acid can make up 18-20% of the total VFA and reaches its highest concentration in high-grain diets. Propionic acid provides energy through conversion to blood glucose in the liver, and is employed in milk lactose or milk sugar synthesis. The rumen microbes also act to synthesize microbial protein from crude protein in the feed rations to produce amino acids. The amino acids in turn produce milk protein.

Starch is a major component of ruminant diets, often comprising over 30% of lactating dairy cow diets and over 60% of diets for beef feedlot finishing diets on a dry matter (“DM”) basis. Then, starch can be fermented to VFA's in the rumen, digested to glucose in the small intestine, or fermented to volatile fatty acids in the large intestine. Degradability of dietary starch affects site of digestion and whole tract digestibility. Site of digestion, in turn, affects fermentation acid production, ruminal pH, microbial yield, and efficiency of microbial protein production. All such factors can affect the productivity of ruminant animals. Many factors affect site of starch digestion in ruminants including DM intake, forage content of the diet, processing, and conservation methods. Grain processing is costly but is often justified economically to increase degradability of starch. High moisture corn grain generally has higher starch degradability than dry corn grain. This is partly because vitreousness of corn endosperm increases with maturity at harvest. Philippeau, C. and B. Michalet-Doreau, “Influence of Genotype and Stage of Maturity of Maize on Rate of Ruminal Starch Degradation,” Animal Feed Sci. Tech. 68: 25-35 (1997). In addition, ensiling corn increases starch degradability. Philippeau, C. and B. Michalet-Doreau, “Influence of Genotype and Ensiling of Corn Grain on in Situ Degradation of Starch in the Rumen,” J. Dairy Sci. 81: 2178-2184 (1999). Stock, R. A., M. H. Sindt, R. Cleale IV, and R. A. Britton, “High-Moisture Corn Utilization in Finishing Cattle,” J. Anim. Sci. 69: 1645 (1991) reported that solubility of endosperm proteins was highly related to moisture level in high moisture corn and solubility increased with time of storage. Endosperm proteins seem to decrease access of starch granules to amylolytic enzymes.

Endosperm type also affects starch degradability, and it is well known that the proportion of vitreous and floury endosperm varies by corn hybrid. Dado, R. G. and R. W. Briggs, “Ruminal Starch Digestibility of Grain from High-Lysine Corn Hybrids Harvested After Black Layer,” J. Dairy Sci. 79 (Suppl. 1): 213 (1996) reported that in vitro starch digestibility (“IVSD”) of seven hybrids of corn with floury endosperm was much higher than that for one yellow dent hybrid. Philippeau, C. and B. Michalet-Doreau, “Influence of Genotype of Corn on Rate of Ruminal Starch Degradation,” J. Dairy Sci. 79 (Suppl. 1): 138 (1996) reported much higher in situ ruminal starch degradation for dent corn compared to flint corn harvested at both the hard dough stage and mature (300 g/kg and 450 g/kg whole plant DM, respectively). Grain (grain refers broadly to a harvested commodity) processing increases the availability of starch in floury endosperm much more than starch in vitreous endosperm (Huntington, 1997). Cells in the floury endosperm are completely disrupted when processed, releasing free starch granules. Watson, S. A. and P. E. Ramstad, “Corn Chemistry and Technology,” Am. Soc. Cereal Chem., St. Paul, Minn. (1987). In contrast, there is little release of starch granules during processing for vitreous endosperm because the protein matrix is thicker and stronger. It is generally assumed that corn with a greater proportion of floury endosperm might have greater starch digestibility and be more responsive to processing.

Neutral detergent fiber (“NDF”) from forage is another important component in many ruminant diets. Forage NDF is needed to stimulate chewing and secretion of salivary buffers to neutralize fermentation acids in the rumen. Increasing the concentration of NDF in forage would mean that less NDF would have to be grown or purchased by the farmer. Thus, crops with higher than normal NDF concentrations would have economic value as a fiber source. However, that value would be reduced or eliminated if the higher NDF concentration resulted in lower digestibility and lower available energy concentrations. Beck et al., WO/02096191, recognized the need for optimizing starch degradability by careful selection of corn having specific grain endosperm type, in view of the ruminal rate of starch degradation, moisture content, and conservation methods used, combined with selection of corn for silage production with specific characteristics for NDF content and NDF digestibility.

Selecting a plant based on its genetics for inclusion in a feed formulation results in inconsistent ruminant animal productivity. For example, selection of a corn hybrid based on its grain endosperm type will yield inconsistent ruminant animal productivity over time.

Various analytical methods are known within the industry for measuring digestibility of starch in grain or fiber in forage. There are in vitro laboratory tests in which, for example, grain from a crop plant is ground into a fine-particle mass, and immersed in an enzyme solution for, e.g., seven hours. The proportion of the starch component of the grain sample that is enzymatically degraded is measured as in vitro starch digestibility (“IVSD”). Similarly, the fiber proportion of a forage crop sample is ground into a mass of fine particles, and immersed in an enzyme solution for a defined time period. The proportion of the fiber component of the forage sample that is enzymatically degraded is measured as NDF digestibility (“NDFd”). Unfortunately, there is no commonly accepted standard for the time period for conducting fiber digestion tests, and different time periods of 12, 24, 30, and even 48 hours are commonly used, thereby influencing the NDFd data results. See Shaver, R. D., “Practical Application of New Forage Quality Tests,” Proceedings of the 6^{th} Western Dairy Management Conference, Reno, Nev. (Mar. 12-14, 2003); Mertens, D. R., “Creating a System for Meeting the Fiber Requirements of Dairy Cows”, J. Dairy Sci. 80: 1463-81 (1997); Shaver, R., “Using NDF Digestibility Information in Dairy Cattle Feeding Program,” 60^{th} Annual; Convention of Virginia State Feed Association and Nutritional Management “Cow” College, Roanoke, Va. (Feb. 22-24, 2006).

Plant breeders are frequently interested in measuring starch and fiber digestion characteristics of the hybrids and varieties that they breed. Food scientists like cereal and oilseed chemists also measure starch and fiber contents of corn, wheat, and oilseed plants in order to determine their suitability for producing food ingredients. Crop farmers may have analyses conducted of the compositional characteristics of their crops growing in their fields to make harvest management decisions. Finally, livestock farmers may choose to obtain analyses of the compositional characteristics of the feed ingredients that their animals consume in order to enhance herd health and productivity.

Use of such laboratory analytical techniques to characterize crop plants can require frequent testing of large number of samples, which can be laborious and time consuming. Therefore, efforts have been made to apply near infrared reflectance spectroscopy (“NIRS”) to the characterization of forage materials. A non-destructive technique based upon the development of prediction equations using historic data for a particular crop hybrid or variety, NIRS has been used to predict the chemical composition of forage plants (e.g., crude protein, starch, and fiber), as well as fiber digestibility. See, e.g., Shenk, J. S. and M. O. Westerhaus, “The Application of Near Infrared Reflectance Spectroscopy (NIRS) to Forage Analysis,” 406-99, Ch. 10 of National Conference on Forage Quality, Evolution, and Utilization Proceedings, University of Nebraska, Lincoln, Nebr. (Apr. 12-15, 1994); Givens, D. I. and E. R. Deaville, “The Current and Future Role of Near Infrared Reflectance Spectroscopy in General Nutrition: a Review, “Aust. J. Agric. Res. 50:1131-45 (1999); Mueller-Harvey, I., “Modern Techniques for Feed Analysis,” Assessing Quality and Safety of Animal Feeds, Food and Agricultural Organization of the United Nations, 1-37 (2004); Agriculture Handbook No. 643, “Near Infrared Reflectance Spectroscopy (NIRS): Analysis of Forage Quality,” United States Department of Agriculture, Agricultural Research Service, 96-103 (1989). While such an NIRS production tool can provide results more quickly than individual laboratory chemistry tests could produce, it is dependent upon the collection of a sufficiently large enough crop data database to enable the development of accurate prediction equations. If done properly, forage NIRS analysis data can be used to predict forage energy content and net energy of lactation of a cow consuming a feed ration containing that forage ingredient. See Lundberg, K. M., P. C. Hoffman, L. M. Bauman, and P. Berzaghi, “Prediction of Forage Energy Content by Near Infrared Reflectance Spectroscopy and Summative Equations,” Professional Animal Scientist, 20: 262-69 (2004); Stallings, C. C., “MILK 2000—Optimizing Yield of Digestible Energy Per Acre from Corn Silage,” Proceedings of the Feed and Nutritional Management Cow College, Virginia Tech, Va. (Jan. 11-12, 2005); Martens, G. C. and R. F. Barnes, “Prediction of Energy Digestibilities of Forages With In Vitro Rumen Fermentation and Fungal Enzyme Systems,” Proc. Int. Workshop on Standardization of Analytical Methodology for Feeds, IDRC-134e, Ottawa, Canada (Unipub. New York, N.Y.: W. J. Pigden et al. ed.) 61-71 (1980).

A number of different visual, physical, and video-based technologist have been applied to the evaluation of grain quality. Wrigley, C. W., “Potential Methodology and Strategies for the Rapid Assessment of Feed Grain Quality,” Aust, J. Agric. Res., 50: 789-805 (1999). NIRS has been used sparingly to measure the compositional characteristics of grain like moisture, protein, starch, oil, and fiber in oilseeds, and moisture and protein in wheat. However, no effort seems to have been made previously to apply NIRS to characterization of starch digestibility of grain samples. See also U.S. Pat. No. 6,844,194 issued to Camerer, III et al. that discloses NIRS testing of whole crop plants for crude protein, fat, and moisture without mention of fiber or starch digestibility.

Therefore, an NIRS-based tool that analyzes the starch and fiber digestibility characteristics of grain and forage samples of a plant for use in real time would be very beneficial. Moreover, such a tool that uses algorithms incorporating such starch and fiber digestibility values and other nutritional characteristics of all the ingredients used in a total mixed ration (“TMR”) to provide a “total ration fermentation index” value for the food ration would be very helpful for proper formulation of feed rations to optimize ruminant animal productivity. Finally an NIRS-based tool that incorporates such starch and fiber digestibility values and other nutritional characteristics of all the feed ingredients incorporated into a TMR to produce a “flash fermentation index” for the feed ration over the first, e.g., two hours of feed digestion would identify specific TMR's that run the risk of causing acidosis in cows which consume such rations.

### SUMMARY OF THE INVENTION

A computer-based system for characterizing in real time the nutritional components of one or more ingredients for a ruminant feed ration, including dry matter, NDF, NDFd, lignified NDF ratio, peNDF, percent starch, IVSD, and particle size for a forage material; and IVSD and particle size for a starch grain material. The system utilizes proprietary NIRS equations based upon prior samplings of a variety of crop species like dual-purpose corn silage, leafy corn silage, brown midrib (“BMR”) corn silage, grass (silage/dry), alfalfa (silage/dry), BMR forage sorghum, normal dent starch grain, floury endosperm starch grain, and vitreous endosperm grain, and applies those equations to current samplings of a corresponding crop to predict in real time the characteristics of such forage or grain material. The real-time characterization system may also utilize the predicted data to calculate a “total ration fermentation index” value that takes into account the total NDFd and IVSD characteristics (including ruminal available starch (“RAS”) and ruminal bypass starch (“RBS”)) of the forage and starch ingredients to be used in a feed ration to ensure that the ration will not contribute too much or too little fermentability to the cow. Thus, using the real-time characterization system enables the proper formulation of a ruminant feed ration and the reformulation of that ration where warranted in the case that the NDFd and IVSD characteristics of the feed components change over time, or the ration ingredient composition changes.

The associated method of the present invention takes into account environmental factors by measuring the starch and fiber degradation characteristics of a variety of genetically different crop plants and grain from crop plants in real time to determine how the crop plants should be blended into a feed formulation that results in optimum productivity of the ruminant animal. It includes providing a feed formulation resulting in optimum ruminant productivity comprising the steps of determining starch digestibility characteristics of a set of crop plant samples comprising grain of the crop plant, developing a prediction equation based on the starch digestibility characteristics, obtaining a grain sample from a crop plant, determining in real time starch digestibility characteristics by NIRS of the sample by inputting electronically recorded near infrared spectrum data from said NIRS into said equation, storing and/or milling said grain on an identity preserved basis, and determining the amount of the crop plant to incorporate into a feed formulation based on the starch digestibility characteristics.

The associated method of the present invention also includes providing a ruminant diet resulting in optimum ruminant productivity comprising the steps of, determining starch digestibility characteristics of grain from genetically different crop plants, determining NDF digestibility (“NDFd”) characteristics of genetically different crop plants for use as forage, developing prediction equations based on the starch digestibility and NDFd characteristics, obtaining grain samples for use as feed supplements and crop plants for use as forage, determining starch and NDFd characteristics by NIRS of the grain samples and the crop plants by inputting electronically recorded near infrared spectrum data relating to the characteristics into the equations and determining the amounts of the grain and the crop plants to incorporate into a feed formulation based on the starch and NDF digestibility characteristics.

The associated method of the present invention further includes providing a ruminant diet resulting in optimum ruminant productivity comprising the steps of, determining in real time starch digestibility characteristics of grain from a crop plants, determining in real time NDFd characteristics of crop plants for use as forage, preserving the grain and the crop plants for use as forage on an identity preserved basis, and determining the amounts of the grain and the crop plants for use a forage to incorporate into a feed formulation based on the starch and NDFd characteristics.

The real-time characterization method of the present invention enhances the energy utilization of a feed formulation by mixing identity preserved grains together in a formulation to obtain a specified degree of rate and extent of digestion of the feed formulation. It determines the quantity of the grain to be used in a feed formulation based on the compatibility and NDFd of a forage source and rate of starch digestion of the grain source. It further determines the quantity of the grain to be used in a feed formulation based on the level of forage NDF and the degree of rate and extent of starch digestion of grain to be used in the feed formulation.

This invention also provides a basis for calculating a “flash fermentation” value or index utilizing the real-time characterizations of the NDFd and IVSD values for the individual components for a feed ration in order to determine whether the ration, due to environmental or other factors, will be too “hot” for the cow and create the risk of reduced meal size, digestive upsets, acidosis and other potential health problems. Finally, through accurate scoring the cows for fermentability, productivity, and stability, dividing them between positive versus negative-response pens, and tailoring the diets for the resulting cows in each pen, the overall productivity and health of the herd can be optimized.

### BRIEF DESCRIPTION OF THE DRAWINGS

In the accompanying drawings:

FIG. 1 is a depiction of a scorecard for a group of cows fed feed formulations in accordance with this invention.

FIG. 2 is a graphical depiction of positive-response cows and negative-response cows penned separately in accordance with this invention for receiving different diets addressing their condition.

FIG. 3 is a schematic illustration of a scheme for moving cows between pens during their progression through the lactation cycle.

### DETAILED DESCRIPTION OF THE PREFERRED EMBODIMENT

A computer-based system for characterizing in real time the nutritional components of one of more ingredients for a ruminant feed ration, including dry matter, NDF, NDFd, lignified NDF ratio, physically effective NDF (“peNDF”), percent starch, IVSD, and particle size for a forage material; and IVSD and particle size for a grain material. The system utilizes proprietary NIRS equations based upon prior samplings of a variety of crop species like dual-purpose corn silage, leafy corn silage, brown midrib (“BMR”) corn silage, grass (silage/dry), alfalfa (silage/dry), BMR forage sorghum, normal dent (“mutt”) starch grain, floury endosperm starch grain, vitreous endosperm grain, and steam-flaked mutt, floury, or vitreous grain sources, and applies those equations to current samplings of a corresponding crop to predict in real time the corresponding characteristics of such forage or grain material. The real-time characterization system may also utilize the predicted data to calculate a “total ration fermentation index” value for a TMR that takes into account the total NDFd and IVSD characteristics (including RAS and RBS), rumen residence time of feed, and other nutritional compositional characteristics like dry matter, NDF, peNDF, and crude protein of the forage and starch ingredients to be used in a feed ration to help the nutritionist deliver an optimally formulated feed ration to the ruminant animal to maximize animal productivity. Finally, the real-time characterization tool can incorporate such total NDFd, IVSD (including RAS and RBS), and other nutritional composition characteristics of the TMR to produce a “flash fermentation index” value that predicts the total fermentability value of the TMR within the first, e.g., two hours after consumption by the ruminant animal to avoid feeding “too hot” of a feed ration to the animal that might cause reduced diet intake size, digestive upsets, reduced feed intake leading to acidosis, or other herd health problems. Thus, using the real-time characterization system enables the proper formulation of a ruminant feed ration and the reformulation of that ration where warranted in the case that the NDFd and IVSD characteristics of the feed components change over time.

For purposes of the present invention, “ruminant animal” means any animal having a multiple-compartment stomach for digesting feed ingredients ruminated by the animal, including but not limited to dairy cows, beef cows, sheep, goats, yaks, water buffalo, and camels. Examples of dairy cows particularly include Holstein, Guernsey, Ayshire, Brown Swiss, Jersey, and Milking Shorthorn cows.

In the context of the present invention, “lactation cycle” means the period of time during which a ruminant animal produces milk following the delivery of a new-born animal.

As used within this application, “milk production” means the volume of milk produced by a lactating ruminant animal during a day, week, or other relevant time period.

For purposes of the present invention, “milk peak” means the highest level of milk production achieved by a ruminant animal during the lactation cycle.

For purposes of this invention, “milk stability” means production by the ruminant animal of milk across the lactation cycle in a manner that approaches the ideal lactation volume each day by achieving optimum milk peak and consistent milk persistence curves for the ruminant animal.

As used within this application, “nutritionist” means an individual responsible for specifying the composition of a feeding ration for a ruminant animal. Such nutritionist can be a dairy farmer, employee of a dairy farm company, or consultant hired by such a farmer or company.

For purposes of this invention, “neutral detergent fiber” (“NDF”) means the insoluble residue remaining after boiling a feed sample in neutral detergent. The major components are lignin, cellulose and hemicellulose, but NDF also contains protein, bound nitrogen, minerals, and cuticle. It is negatively related to feed intake and digestibility by ruminants.

As used within this application “NDF digestibility” (“NDFd”) means the amount of NDF that is fermented by rumen microbes at a fixed time point and is used as an indicator of forage quality. Common endpoints for fermentation are: 24, 30, or 48 hours. NDFd is positively associated with feed intake, milk production, and body weight gain in dairy cattle.

For purposes of this invention, “lignified NDF” means the fraction of NDF that is protected from fermentation by its chemical and physical relationship with lignin. It is commonly referred to as indigestible NDF and is often estimated as (lignin×2.4).

As used within this application, “effective fiber,” more commonly referred to as “physically effective fiber” (“peNDF”), means the fraction of NDF that stimulates rumination and forms the digesta mat in the rumen. It is measured as the fraction of particles retained on the 1.18-mm screen when a sample is dry sieved.

For the present invention, “dry matter intake” means the amount of feed (on a moisture-free basis) that an animal consumes in a given period of time, typically 24 hours. Calculated as feed offered-feed refused (all on a moisture-free basis).

For purposes of the present invention, “volatile fatty acids” (“VFA”) are the end product of anaerobic microbial fermentation of feed ingredients in the rumen. The common VFA's are acetate, propionate, butyrate, isobutyrate, valerate, and isovalerate. The VFA's are absorbed by the rumen and used by the animal for energy and lipid synthesis.

As used within this application, “RAS” means ruminal available starch.

In the context of the present invention, “RBS” means ruminal bypass starch.

The real-time characterization system and associated feeding method and feed composition of the present invention is discussed within this application for a dairy cow. However, it should be understood that this invention can be applied to any other ruminant animal including ruminants that are not used to produce milk like beef steers used for meat production.

A number of different variables impact the effective delivery to and utilization by the dairy cow of nutritional ingredients contained in a feed ration. Called the “GELT Effect” by Applicant, the variables include genetics, environment, location, and traits. The specific genetics of the cow will directly influence its ability to digest and absorb the nutritional ingredients. Likewise, the specific genetics of the forage and grain components of the feed components can directly influence their nutritional content of carbohydrates, protein, and fiber. Therefore, corn genetics used for corn silage production have a significant range of NDF content, NDFd, and percent starch content. Likewise, grain genetics have a wide range of oil, protein, starch composition, and rate and extent of starch digestibility. Thus, the seed genetics determines the potential of each forage and grain quality trait to deliver nutrition to the cow. Failure to use appropriate agronomic inputs (e.g., fertilizers, herbicides, fungicides, pesticides) and levels thereof can also have a deleterious effect upon the quality trail characteristics of the resulting crop grown from the seed.

The environment and weather conditions under which a crop is grown is another key source of variability. The weather is considered an uncontrollable event. No one growing season is the same from one year to the next in terms of temperature and moisture. This directly affects and adds a high degree of variation to forage production, forage quality, and starch digestibility that can create subsequent inconsistencies in a dairy cow's performance. For example temperature and rainfall patterns during a growing season can affect the level of fiber (NDF), the amount, and the effect of lignin on fiber digestibility (NDFd). This subsequently can affect how a forage “feeds,” and can have an increase or decrease effect on dry matter intake (DMI) and energy intake with dairy cows, especially cows that are limited by fill and in early lactation.

Starch digestibility within the kernels of a corn hybrid chopped for silage and corn grain used for energy supplementation can also be variable by a growing season environment. Both the content of starch and the rate and extent of digestion can be altered. Thus, supplement grain added to a diet and the corn grain within corn silage can positively and negatively affect dairy cow productivity. Hence the environment determines the level and range of each forage and grain quality trait.

The temperature and other feeding conditions can also directly influence the cow's willingness or ability to intake dry matter contained in feed rations. Thus, this environmental variation makes it almost impossible to predict and implement a feed programming strategy for a dairy cow in a given production year, or design a cropping or ingredient purchasing program for growing or procuring forage and grain feed ingredients without utilization of some type of real-time adjustment mechanism to account for this uncontrolled variation factor.

Specific harvesting techniques can also have a deleterious influence upon the nutritional content of the feed ingredient. Poor storage techniques (e.g., packing and storage) can also adversely impact the nutritional value of grain, forage or silage. Sampling protocols and laboratory testing errors arising during the analysis of the nutritional profile of a feed ingredient can interfere with construction of an appropriate feed ration. Moreover, the inoculants used to facilitate forage fermentation to produce silage, and preservatives for silage and grain storage can adversely impact the nutritional traits of the silage or grain product. Harvest management techniques therefore determine the net of each forage and grain quality trait. Of course, poor formulation of the feed ration can also affect the proper delivery of nutritional values to the dairy cow.

Therefore, it is important to appreciate that no two forage or grain samples are exactly the same in nutritional content, even if grown from the same seed variety or hybrid, and the nutritional content of different varieties and hybrids will probably vary significantly—all because of this GELT Effect.

A feeding method associated with the real-time characterization system of the present invention is disclosed in Applicant's U.S. Ser. No. 11/494,312 filed on Jul. 27, 2006, and U.S. Ser. No. 11/881,490 filed on Jul. 27, 2007, both of which are incorporated hereby in their entirety.

A feed delivery system associated with the real-time characterization system of the present invention is disclosed in Applicant's U.S. Ser. No. 11/494,312 filed on Jul. 27, 2006, and U.S. Ser. No. 11/881,483 filed on Jul. 27, 2007, both of which are incorporated hereby in their entirety.

I. Interactive Effect of a Plant Crop and the Environment

Six corn hybrids were grown in duplicate plots in 3 locations in the 1999 growing season. Locations were East Lansing, Mich.; Lincoln, Nebr.; and University Park, Pa. The six hybrids included several endosperm types: 1 floury, 1 opaque-2, 1 waxy, 1 dent, and 2 flint hybrids. Plots were 32 rows wide by 400′ long (30″ rows).

Each field was monitored once per week beginning September 15th. Following physiological maturity at black layer (BL), grain dry matter (DM) was determined weekly for all plots. Grain was harvested at 60%, 70% and 80% DM from all plots. To minimize probability of cross-pollination, ten ears were harvested from each of the middle two rows of each plot (rows 16 and 17) for a total of 20 ears. Ears were not harvested from plants within 100′ of the ends of the 400′ long plots and were taken approximately every 20′ along the 200′ remaining. Grain was shelled from the ears by hand. A 500 g sample of grain was taken for determination of DM, vitreousness, and density. The remainder of the grain was rolled and ensiled in duplicate 4″×12″ PVC experimental silos. An additional sample (0.5 kg) was taken as a 0 time sample.

One of each duplicate silo from each plot and maturity was opened at 35-d after harvest and the other was opened at 120-d after harvest. Contents of silos were frozen for subsequent analysis. Samples were ground with dry ice (Wiley mill, 1-mm screen) before analysis. In vitro starch degradation was determined after incubation for 7 h in buffered media with 20% rumen fluid.

All samples were characterized for starch, sugars, ether extract, crude protein content, and protein solubility in sequential buffers. Samples of intact kernels taken at harvest were analyzed for vitreousness and density in ethanol (Philippeau and Michalet-Doreau, 1997). Samples taken after rolling that were not ensiled (n=72) were dried at 55° C., dry sieved and analyzed for particle size. Starch degradability, also referred to herein as digestibility, was determined by in vitro starch digestion with rumen microbes and measuring starch disappearance over time. Other methods for measuring starch digestion known include gas production, in vitro starch disappearance using enzymes, and in situ starch digestion.

Vitreousness of endosperm for the hybrids tested ranged from 4 to 62%. Table 1 shows that starch digestion was affected by the corn hybrid (49.8 to 60.3%, P<0.001). Table 2 shows that starch digestion increased with moisture content (46.0 to 65.8%, P<0.001). Table 2 also shows that starch digestion was affected by ensiling (0 days vs. 35 days and 120 days, 46.3% vs. 59.3%, P=0.001), and time of ensiling (35 days vs. 120 days, 57.4% vs. 61.25%, P<0.001).

Table 3 establishes that starch digestion is dependent upon several interactions between hybrid and the environment. A p-value of less than 0.05 is significant for single sources, whereas a p-value of less than 0.1 is significant for interactions between sources. Thus, location, moisture, hybrid, day, all had a significant effect on starch digestibility. The results show that the interactions of Moisture×Day, Moisture×Location, Moisture×Hybrid, and Hybrid×Location were all significant. For example, the effect of the hybrid on starch digestibility changed at different moisture levels. Table 3 also shows that a hybrid's effect on starch digestibility depends upon the location where it was grown and, therefore, starch digestibility of a particular hybrid varies across different locations. Tables 4, 5, 6 and 7 show the data for the interaction between hybrids and their growth environments and the effect these interactions have on starch digestibility of the hybrids. For example, Table 4 shows that the effect of Day×Moisture on starch digestibility is disproportionate to either environmental factor alone. Likewise, the interactive effects of Moisture—Location (Table 5), Moisture—Hybrid (Table 6), and Hybrid×Location (Table 7) all show strong interactive effects on starch digestibility.

TABLE 1
Corn hybrid means for in-vitro starch digestibility (IVSD),
averaged over three stages of maturity, 3 post harvest intervals, 2
plots per location and 3 locations.
Effect of Hybrid on IVSD

| Hybrid | IVSD |
|---|---|
| N4342 wx | 49.8 |
| 6409 GQ | 50.9 |
| W1698 | 54.3 |
| N4640Bt | 57.5 |
| NX7219 | 57.5 |
| SL-53 | 60.3 |
| SE-1.26 | |

TABLE 2
IVSD means for three moistures and three storage intervals.

| | |
|---|---|
| Effect of Moisture | Effect of |
| % on IVSD | Day on IVSD |

| Moisture % | IVSD | Day | IVSD |
|---|---|---|---|
| 20 | 46.0 | 0 | 46.3 |
| 30 | 53.1 | 35 | 57.4 |
| 40 | 65.8 | 120 | 61.2 |

| | |
|---|---|
| SE = 1.03 | SE = 0.84 |

TABLE 3
Levels of significance for pertinent sources of variation in IVSD.
Treatment Effects on IV Starch Digestibility

| | Degrees of | |
|---|---|---|
| Source | Freedom (DF) | Prob > F |
| Location | 2 | 0.19 |
| Moisture | 2 | <0.0001 |
| Hybrid | 5 | <0.0001 |
| Day | 2 | <0.0001 |
| Moisture × Day | 4 | <0.0001 |
| Moisture × Location | 4 | 0.07 |
| Moisture × Hybrid | 10 | 0.08 |
| Hybrid × Location | 10 | 0.08 |

TABLE 4
IVSD Moisture × Day interaction means for three moistures and
three storage intervals
Moisture × Day

| | Day | | |
|---|---|---|---|
| Moisture % | 0 | 35 | 120 |
| 20 | 43.9 | 46.7 | 47.5 |
| 30 | 44.1 | 55.5 | 59.7 |
| 40 | 50.8 | 70.1 | 76.4 |

TABLE 5
IVSD Moisture × Location interaction means for three moistures
and three locations
Moisture × Location

| | Location | | |
|---|---|---|---|
| Moisture % | #1 | #2 | #3 |
| 20 | 46.1 | 46.8 | 45.2 |
| 30 | 51.5 | 54.6 | 53.3 |
| 40 | 63.8 | 63.2 | 70.3 |

TABLE 6
IVSD Moisture × Hybrid interaction means for three moistures
and six hybrids
Moisture × Hybrid

| | Moisture % | | |
|---|---|---|---|
| Hybrid | 20 | 30 | 40 |
| N4342wx | 41.7 | 44.3 | 63.4 |
| 6409 GQ | 40.9 | 52.8 | 58.9 |
| W1698 | 44.6 | 52.7 | 65.8 |
| N4640Bt | 47.8 | 57.8 | 65.0 |
| NX7219 | 49.9 | 52.5 | 70.2 |
| SL-53 | 51.4 | 58.6 | 71.2 |

TABLE 7
IVSD Hybrid × Location interaction means for six hybrids and
three locations. The number in parentheses is the rank of the
hybrid within location.
Hybrid × Location

| | Location | | |
|---|---|---|---|
| Hybrid | #1 | #2 | #3 |
| N4342wx | 51.1 (4) | 51.4 (5) | 46.9 (6) |
| 6409 GQ | 49.7 (6) | 50.1 (6) | 52.8 (5) |
| W1698 | 50.0 (5) | 54.2 (4) | 58.7 (2) |
| N4640Bt | 56.2 (3) | 61.2 (2) | 53.2 (4) |
| NX7219 | 56.4 (2) | 58.9 (3) | 57.3 (3) |
| SL-53 | 59.4 (1) | 61.5 (1) | 60.2 (1) |

II. Measurement of Feed Ingredient Characteristics

The current inventory of forage and grain ingredients on farm, as well as any new forage and grain crops that may be planted by the dairy farm need to be characterized in real time. A representative sample of each crop hybrid or variety in each field is obtained and scanned using NIRS at the wavelengths required by a corresponding prediction equation previously developed. This NIRS analysis is done in a laboratory or in the field using a portable NIRS instrument. It is desirable that the methods used to measure these traits are relatively quick, e.g., in real time. “Real time,” as used within this application, refers to obtaining the starch and fiber digestibility results and other composition characterization results within 48 hours from when the samples are obtained and tested, more preferably within 24 hours from when the samples are obtained and tested.

For purposes of this invention, the fiber digestion (“NDFd”) characteristics of the crop forage portion of the hybrid or variety is predicted using the prediction equation for that hybrid or variety. Moreover, the starch digestibility (IVSD) characteristics of the grain and forage portions of the hybrid or variety are predicted using that set of equations. The starch characteristics are then used to determine the ruminal available starch (RAS) and ruminal bypass starch (RBS) of that hybrid or variety. The relevant compositional characteristics for the hybrid or variety that should be predicted using corresponding equations include dry matter percentage, NDF percentage, peNDF, and crude protein percentage.

These NIRS-derived characteristic predictions should be conducted upon each component of a feed ration. This might include without limitation: brown midrib corn silage, dual purpose corn silage and/or leafy corn silage as the primary forage source; haylage or dry hay as the secondary forage source; and high-moisture mutt corn, steam-flaked mutt or specific endosperm corn, dry floury corn starch and/or dry vitreous corn starch as the grain source. By using the NIRS-based real-time characterization tool of the present invention to predict the starch digestibility (including RAS and RBS), fiber digestibility, and compositional characteristics of each ingredient for a feed ration, a more accurate and complete understanding of the nutritional load to be delivered by the TMR to the dairy cow can be obtained.

The NIRS method includes obtaining a set of crop plant samples with known characteristic such as starch and fiber degradability. These characteristics are measured according to the IVSD and NDFd measurement methods described below. Other starch and NDFd measurement methods known in the art can be used as well. These crop plant samples are scanned in the near infrared spectrum. Reflectance in the near-infrared spectrum is then recorded. A prediction equation for each trait is developed by regressing the known measured characteristics on reflectance across wavelengths for each set of samples.

For each trait, the prediction equation is validated by predicting the characteristic of interest for an independent set of samples. According to the present invention, the measured characteristics of interest in grain include without limitation: % IVSD in the grain, corn silage, HMC or dry corn, and particle size. These values reflect the rate and extent of ruminal starch digestibility at a specified digestion period, usually 7 hours. IVSD should be measured at different particle sizes, such as 6 mm, 4 mm, 2 mm, 2 UD, and 1 UD. For the forage sources, characteristics of interest include without limitation dry matter content, NDF, fiber digestibility (NDFd), lignin content, peNDF, in vitro whole plant digestibility (IVTD), corn silage starch digestibility (IVSD-CS), corn silage particle size at different lengths of chop (peNDF) and conservation processing methods. Finally, separate equations should be developed for different crop species to be used with the feed rations, including but not limited to dual-purpose corn, leafy corn, BMR corn, grass (silage/dry), alfalfa (silage/dry), and BMR forage sorghum, normal dent corn starch grain, mutt corn starch grain, floury endosperm starch grain, and vitreous endosperm starch grain. Furthermore, prediction equations can predict the fiber or starch digestibility characteristics of the forage or starch component for different particle sizes. Of significant value is the fact that an “as-is” wet crop sample can be evaluated in real time without the need to dry and grind it as conventional laboratory NIRS instruments require.

Near-infrared reflectance spectroscopy (NIRS) is a nondestructive, instrumental method for rapid, accurate, and precise determination of the chemical composition of forages and feedstuffs. NIRS is an accepted technology for feed and forage analysis, and industrial uses. NIRS has several distinct advantages: the speed of analysis, non-destructive analysis of the sample, simplicity of sample preparation, and several analyses can be completed with one sample. Since NIRS analysis is relatively simple to perform, operator-induced errors are reduced (Shenk and Westerhaus, 1994).

To measure starch degradability in vitro, a set of crop plant samples comprising a number of genetically different crop plants are analyzed for starch concentration before and after incubation in media inoculated with rumen fluid containing ruminal microbes for various lengths of times. Starch degradability is calculated as the amount of starch that disappeared as a percent of the total starch in the sample for each time point of interest. Starch concentration can be determined by analysis of glucose concentration before and after hydrolysis using commercially available analysis kits. Glucose concentration may be determined enzymatically using glucose oxidase method or by high performance liquid chromatography. For general methods of measuring feed digestibility in vitro, see Goering, H. K. and P. J. Van Soest, “Forage Fiber Analysis: Apparatus, Reagents, Procedures, and Some Applications,” USDA-ARS Handbook 379, U.S. Govt. Print. Office, Washington, D.C. (1970). An alternative method is to incubate feed samples in porous bags in the rumen of cattle or sheep. (Philippeau and Michalet—Doreau, 1997).

To measure fiber digestibility in vitro, dried plant tissues were ground with a Wiley® mill to pass a 1 mm screen. In vitro true digestibility (IVTD) and in vitro neutral detergent fiber digestibility was determined using 0.5 g samples using a modification of the method of Goering and Van Soest (1970) with an incubation time representing the rumen residence time of the animal of interest such as 30 h. Undigested IVTD residue was subjected to the neutral detergent fiber (NDF) procedure (Goering and Van Soest, 1970). A modification of the NDF procedure was the treatment of all samples with 0.1 ml of alpha-amylase during refluxing and again during sample filtration, as described by Mertens, D. R., “Neutral Detergent Fiber,” Proc. National Forage Testing Association Forage Analysis Workshop, Milwaukee, Wis., A12-16 (May 7-8, 1991). Alpha-amylase was assayed for activity prior to use, according to Mertens (1991). NDF digestibility (dNDF) for each sample was computed by the equation: 100*[(NDF−(100−IVTD))/NDF].

Accuracy of the laboratory values for defining the forage quality parameters of the forage and the starch digestibility profile of the grains is paramount to value creation from the invention. To maximize the synergy of the forage and grain specs, the accuracy of the forage template to capture the forage synergy of the forage sources, and to properly develop the feeding template requires accurate characterization. It is therefore important to use only analytical laboratories that are certified by the National Forage Testing Association (NFTA) to maintain the accuracy and consistency of the characterization process.

The invention requires an approved certified lab to characterize both forage and grains to establish a historic baseline for each characterized trait. This baseline can be used to determine the hybrid genetic effect and the environmental effect within a given growing season on the forage quality traits and the potential feeding value of both forages and grains used in the nutritional template. Accurate adjustments can then be made to the nutritional template to maintain the accuracy of the resulting feeding template for each stage of dairy cow production.

The same real-time characterization process is used in the genetic development of superior forage and grain genetics necessary for the feed ingredients. Real-time characterization measures the direction, progress and level of trait enhancement of the breeding process. It also is used as a database development tool for screening and identifying the top performing genetics for invention application.

According to the present invention, databases are developed relating the NIR spectrum to the starch and fiber degradability and compositional characteristics of a number of genetically different crop plants. The NIR spectrums of a given crop plant such as corn, soybean, or alfalfa are used to assess the crop plant's starch and fiber degradability and compositional characteristics. The NIRS method may be applied to various feed crops and the traits of those crops. NIRS requires a calibration to reference methods (Shenk and Westerhaus, 1994). Each constituent requires a separate calibration, and in general, the calibration is valid for similar types of samples.

The NIRS method of analysis is based on the relationship that exists between infrared absorption characteristics and the major chemical components of a sample (Shenk and Westerhaus, 1994). The near infrared absorption characteristics can be used to differentiate the chemical components. Each of the significant organic plant components has absorption characteristics (due to vibrations originating from the stretching and bending of hydrogen bonds associated with carbon, oxygen and nitrogen) in the near infrared region that are specific to the component of interest. The absorption characteristics are the primary determinants of diffuse reflectance, which provides the means of assessing composition. The diffuse reflectance of a sample is a sum of the absorption properties combined with the radiation-scattering properties of the sample. As a consequence the near infrared diffuse reflectance signal contains information about sample composition. Appropriate mathematical treatment of the reflectance data will result in extraction of compositional information. Osboume, B. G., T. Fearn, and P. H. Hindle, Practical NIR Spectroscopy With Applications in Food and Beverage Analysis, Longman Scientific and Technical, Essex, England (1986). The most rudimentary way to illustrate this would be to measure the reflectance at two wavelengths, with one wavelength chosen to be at a maximum absorption point and the other at the minimum absorption point, for the compositional factor to be analyzed. The ratio of the two reflectance values, based on determination of two samples, can be associated, by correlation, to the concentration of the specific compositional factor in those samples. By use of the correlation relationship, an equation can be developed that will predict the concentration of the compositional factors from their reflectance measurements (Osbourne et al., 1986).

Spectra can be collected from the sample in its natural form, or as is often the case with plants or plant parts, they are ground, typically to pass through a 1-mm screen. NIR reflectance measurements are generally transformed by the logarithm of the reverse reflectance (log (1/R)), Hruska, W. R., “Data Analysis: Wavelength Selection Methods, Near-Infrared Technology in the Agricultural and Food Industries (American Association of Cereal Chemists, St. Paul, Minn.: P. Williams and K. Norris ed.) 35-6 (1987), other mathematical transformations known in the art may be used as well. Transformed reflectance data are further mathematically treated by employment of first- or second-derivatives, derivatives of higher order are not commonly used (Shenk and Westerhaus, 1994).

The calibration techniques employed are multiple linear regression (“MLR”) methods relating the NIR absorbance values (x variables) at selected wavelengths to reference values (y values), two commonly used methods are step-up and stepwise regression (Shenk and Westerhaus, 1994). Other calibration methods are principal-component regression (“PCR”), Cowe, I. A. and J. W. McNicol, “The Use of Principal Components in the Analysis of Near Infrared Spectra, Applied Spectroscopy 39:257-66 (1985), partial least-squares regression (“PLS”), Martens, H., and T. Naes, Multivariate Calibration (John Wiley and Sons, New York, N.Y. (1989), and artificial neural networks (“ANN”), Naes, T., K. Kvaal, T. Isaksson, and C. Miller, “Artificial Neural Networks in Multivariate Calibration,” Journal of Near Infrared Spectroscopy 1: 1-12 (1993).

The methods of calibration equation differ depending on the regression method used. The procedure when using MLR is to randomly select samples from the calibration population, exclude them from the calibration process and then use them as a validation set to assess the calibration equation. Windham, W. R., D. R. Mertens, F. E. Barton II, “Supplement 1. Protocol for NIRS Calibration: Sample Selection and Equation Development and Validation,” Near Infrared Reflectance Spectroscopy (NIRS). Analysis of Forage Quality, USDA Agricultural Handbook No. 643, Washington, D.C. 96-103 (1989). The method of equation validation used for PCR or PLS regression is cross-validation, which involves splitting the calibration set into several groups and conducting calibration incrementally on every group until each sample has been used for both calibration and validation. Jackson, J. E., A User's Guide to Principal Components (John Wiley and Sons, New York, N.Y. (1991); Martens and Naes, 1989; Shenk and Westerhaus, 1994.

In this instance, NIRS involves the collection of spectra for a set of samples with known characteristics. The spectra is collected from grain kernels, or other plant parts, and mathematically transformed. A calibration equation is calculated using the PLS method, other regression methods known in the art may be used as well. Criteria used to select calibration equations are low standard errors of calibration and cross validation and high coefficients of multiple determinations.

This tool can also be used to measure quality trains for crop plants other than NFDd and IVSD, such as dry matter %, NDF, peNDF, oil content, and crude protein.

The real-time characterization system of the present invention is a computer-based tool. It comprises a general programmable computer having a central processing unit (“CPU”) controlling a memory unit, a storage unit, an input/output (“I/O”) control unit, and at least one monitor. The computer operatively connects to a database, containing, e.g., dry matter, NDF, NDFd, IVSD, particle size, etc. data for a variety of hybrids and varieties for a variety of crop plants. It may also include clock circuitry, a data interface, a network controller, and an internal bus. One skilled in the art will recognize that other peripheral components such as printers, drives, keyboards, mousse and the like can also be used in conjunction with the programmable the computer. Additionally, one skilled in the art will recognize that the programmable computer can utilize known hardware, software, and the like configurations of varying computer components to optimize the storage and manipulation of the data and other information contained within the real-time characterization tool.

An NIRS reflectance apparatus is used to measure the reflected wavelength of crop samples, and the resulting NIRS data is stored in the database. A software program may be designed to be an expression of an organized set of instructions in a coded language. These instructions are programmed to interact with proprietary prediction equations stored in the memory. When a crop sample in subjected to NIRS analysis in real time, the resulting NIRS data is used by the prediction equations to predict the actual true value of the associated characteristics of the real-time crop sample. As mentioned above, the prediction equations can further predict the fiber or starch digestibility of the forage or grain material at different particle sizes, which can be of great assistance in formulating feed rations.

The computer system on which the system resides may be a standard PC, laptop, mainframe, handheld wireless device, or any automated data processing equipment capable of running software for monitoring the progress of the transplantable material. The CPU controls the computer system and is capable of running the system stored in memory. The memory may include, for example, internal memory such RAM and/or ROM, external memory such as CD-ROMs, DVDs, flash drives, or any currently existing or future data storage means. The clock circuit may include any type of circuitry capable of generating information indicating the present time and/or date. The clock circuitry may also be capable of being programmed to count down a predetermined or set amount of time. This may be particularly important if a particular type of tissue needs to be refrigerated or implanted in a predetermined amount of time.

The data interface allows for communication between one or more networks which may be a LAN (local area network), WAN (wide area network), or any type of network that links each party handling the tissue. Different computer systems such as, for example, a laptop and a wireless device typically use different protocols (i.e., different languages). To allow the disparate devices to communicate, the data interface may include or interact with a data conversion program or device to exchange the data. The data interface may also allow disparate devices to communicate through a Public Switched Telephone Network (PSTN), the Internet, and private or semi-private networks.

III. Applying the Real-Time NIRS Prediction Data to Feed Formulations

The real-time NIRS data obtained above for predicting the starch digestibility, fiber digestibility, and nutritional composition characteristics of various crops can be used in many ways. First, crops about to be harvested are analyzed for starch and fiber degradation and compositional characteristics before harvest to provide information needed for harvesting decisions. A representative sample of each field is obtained and scanned using an NIR spectrophotometer at the wavelengths required by the prediction equation previously developed. Starch and/or fiber digestion and compositional characteristics of the plants in each field are predicted using this equation. Information provided is used to make harvest decisions such as the moisture concentration at harvest and particle size to grind for high moisture grain and the conservation method (high moisture grain or dry grain). This gives additional control over the resulting feed consumed by cattle and sheep, which helps optimize energy intake and nutrient utilization. The NIRS analysis is done in a laboratory or in the field using a portable NIRS instrument.

Second, stored feed samples are screened for starch and fiber digestibility and compositional characteristics to provide information to formulate diets for optimal energy intake and nutrient utilization. Feeds with highly degradable starch are limited in diets to prevent ruminal acidosis, lower fiber digestibility and efficiency of microbial protein production, and decreased energy intake. Feed with low starch degradability is limited to optimize microbial protein production, nutrient utilization, and energy intake.

To this end, this invention includes a “total ration fermentation value” that constitutes a series of interrelated calculations for evaluating the nutritional effectiveness of the feed ration, and its ability to safely deliver the optimum nutritional value to the dairy cow for the pertinent production stage. Constituting an aggregation of the IVSD and NDFs predicted values obtained by the real-time NIRS characterization tool described above, it takes into account the total digestibility of the feed ration, compiling the pounds of digestible fiber contributed by the forage source(s), and the pounds of digestible starch contributed by the grain and forage sources for each and every forage, grain, and other constituents used in the TMR. This total ration fermentation value should also take into account the residence time of the ration within the cow's rumen system, as well as other relevant variables that affect ration fermentability like dry matter percentage, NDF percentage, peNDF, crude protein percentage, and particle size for the TMR. The resulting total ration fermentation value characterizes the total starch digestion, fiber digestion, and other nutritional elements contributed by the feed ration to the dairy cow that consumes it. This holistic approach is much more accurate than the traditional approach used by the animal feed industry to focus upon only one or a couple of factors that influence animal feed intake and nutrition. A range should be specified for this total ration fermentation value within the nutritional template for each stage of production of the cows. By checking the NDFd and IVSD values of the various forage and grain starch ingredients used within the feed ration using the real-time characterization tool on a periodic basis, and plugging these values into the total digestibility equation, the nutritionist can determine whether the GELT Effect has caused one or more of the feed ingredients to provide too much or too little fiber and starch digestibility to the cow that is fed the feed ration. The index can also be used to validate the optimum and accurate feed formulation of the TMR, or catch errors in the formulation process at the mixer wagon before the diet is fed to the cow.

Thus, this total ration fermentation value helps the nutritionist to maximize the productivity of the dairy cow. For ease of use, this total ration formulation value may be translated to a simple index, such as a 1-5 scale, to intuitively characterize the relative fermentation of the feed ration when consumed by the cow over the ensuing time period within the rumen, such as 24 hours.

Third, the nutritionist can use this total ration fermentation value to determine whether the feed formulation used to calculate it needs to be modified. Accordingly, the NDFd and IVSD values should be measured for the individual feed components. This data will tell the nutritionist which specific ingredients are contributing the fiber and starch digestibility to the feed ration. For different stages of production, the cow may need different levels of NDFd and IVSD.

Next, the relative ruminal available starch (“RAS”) and ruminal bypass starch (“RBS”) values should be calculated to see whether the RAS/RBS ratio is within the range specified within the nutritional template. By controlling the RAS/RBS ratio, maximum healthy milk production and milk components (e.g., milk fat, milk protein) may be obtained.

Finally, by comparing the total ration fermentability, individual component digestibilities, and dry matter, NDF, NDFd, IVSD, and RAS/RBS ratio values for the total diet against the corresponding values specified within the nutritional template, the nutritionist can quickly and accurately determine in real time through this tool whether the feed ration ingredients need to be adjusted or modified to bring the diet into conformity with the specifications during the production stage. The specific feed ingredients used might have changed significantly due to environmental or other factors, so that it no longer provides the required nutritional and energy load to the animal. Alternatively, the fiber or starch content of the diet, the IVSD or NDFd values of one or more of the ingredients might have unexpectedly increased to the point that the TMR can cause digestive upsets leading to sub-clinical acidosis, or acidosis in the animal's rumen, thereby leading to reduced feed intake in the short term, and health issues or even death in the longer term.

Not only can use of this total ration fermentation value for the TMR, along with the comparative measurements of NDFd, IVSD, RAS/RBS values for the individual ingredients in accordance with this invention lead to enhanced milk production and stability, but also it can save the cows from serious health issues suffered from feed rations that are too “hot” because individual feed components exhibited unexpectedly high digestibility. This NIRS analysis is done in a laboratory or in the field using a portable NIRS instrument. It is desirable that the methods used to measure these traits are relatively quick, e.g., in real time. Real time refers to obtaining the starch and fiber digestibility results within 48 hours from when the samples are obtained and tested, and more preferably within 24 hours from when the samples are obtained and tested.

Fourth, a “flash fermentation” value or index can be calculated for the TMR of a specific feed ration. Using the real-time characterization values for the specific feed components to be used in the fee ration, the algorithm can predict the total fermentation energy that will be released within the cow's rumen during the short initial time after consumption of the feed ration. The calculation will focus upon total NDFd provided by the forage components, total IVSD provided by the forage and grain components, residence time, and the influence upon fiber digestibility and starch digestibility posed by other feed factors such as dry matter percentage, NDF, peNDF, crude protein, and particle size. For purposes of this invention, the residence time focus for this flash fermentation value should necessarily be short in scope—1.5-3 hours in duration, preferably 2 hours in duration. In this manner, feed rations that are “too hot” can be identified by the nutritionist before they are fed to the cows to prevent reduced meal size, digestive upsets, acidosis and other potential health problems. These effects caused by overly hot feed rations often are not readily apparent to the nutritionist or dairy farmer until it is too late. For instance, cows often eat from the same feed trough within the same pen. It may not be obvious that one cow in particular is eating too little until it shows up in consistently reduced milk production and stability over a period of several days. At this point, the dairy farmer has lost a large volume of potential milk production and quality, and it may take time and expensive care to nurse the sick cow back to health. For purposes of this invention, this flash fermentation value can be translated to a simple index, such as a 1-5 scale, so that it can be quickly and easily used by the nutritionist, just like a chemical ph test.

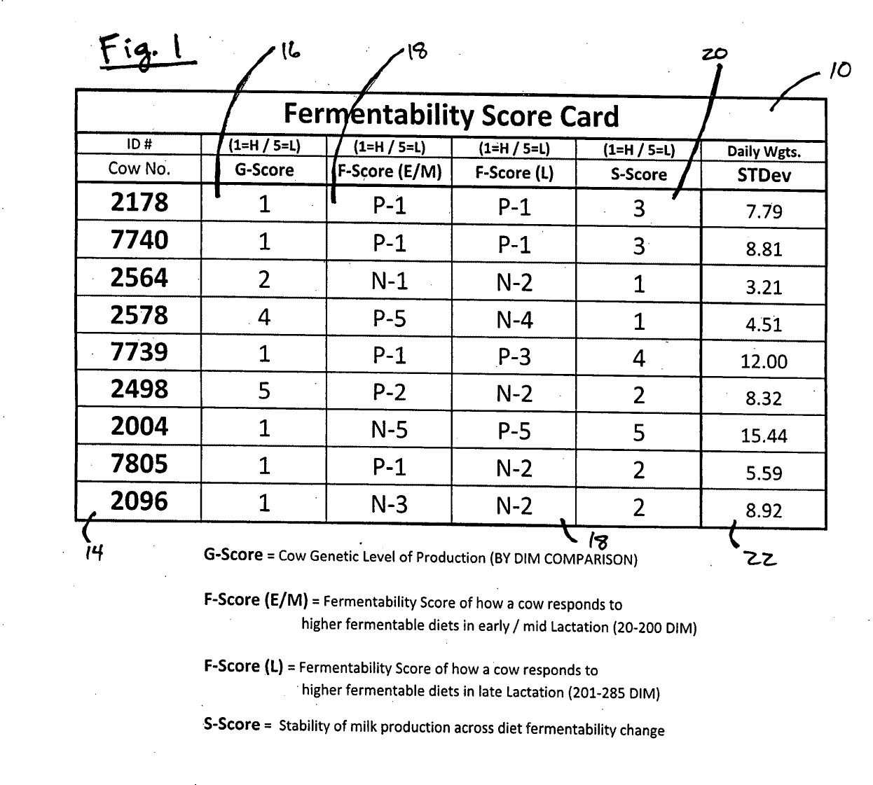

Fifth, it is important to understand that just as cows have varying nutritional requirements between the different production stages of the lactation cycle, no two cows are exactly alike, even within the same production cycle. To this end, the cows under this invention are ideally scored on an individual basis. Each cow's response to the specific feed diet is evaluated on a comparative basis for milk production, milk stability, and fermentability response. As exemplified in FIG. 1, a diet scorecard 10 can be produced in accordance with this invention showing for each cow 12 her identification number 14; “G-Score” 16 for production on a 1-5 scale where “1” is high and “5” is low; “F-Score” 18 for fermentability response within the production state (E/M=early/mid lactation stage; “L”=late lactation stage); “S-Score” 20 for milk stability; and standard deviation from her mean milk production 22 over, e.g., seven days of milking. For fermentation score 18, each cow is also rated as exhibiting a positive “P” response to the feed diet fermentability or negative “N” response thereto. In this manner, the nutritionist is provided valuable information for characterizing for each cow not only her degree of response to a fermentable diet (and ability to consume it without health issues), but also her relative milk production (including on a “fat corrected basis” to take into account the fat and protein portions of her milk) and stability for producing such volume and quality of milk over time. This cow scoring tool enables individual cows to be separated from each other on the basis of fermentability, production, and stability even within the same lactation stage, instead of treating all cows the same when formulating feed rations, as is typical within the dairy industry.

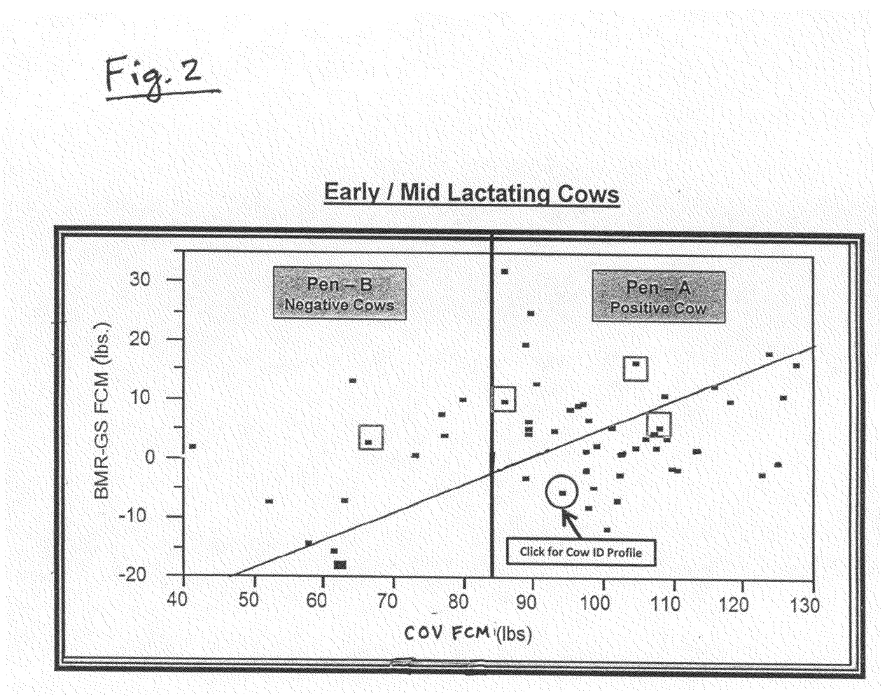

In order to assist with this cow differentiation, the cows may be fed a “challenge diet” featuring an elevated level of fermentability. Such a challenge diet can magnify the cows that react positively to the diet versus those that react negatively to such diet. The positive-response cows should be moved to a Pen A, while the negative-response cows are moved to a Pen B. The feed ration for the positive and negative-response cows can then be reformulated to address their group needs: maximizing the productivity of the high-producing, stable Pen A cows, while addressing the health needs of the Pen B cows. The Pen A and Pen B cows can be plotted graphically, as depicted in FIG. 2 in terms of their positive or negative response to a fermentable diet as a function of their milk production. This can be done on a daily or weekly basis to help the nutritionist detect individual cows undergoing a change in diet response, which might warrant being moved to the other pen. Individual positive response cows can be moved to the negative response B pen, as needed. This tool can also help to determine whether the positive-response cows can handle an even more fermentable diet in order to optimize their productivity. In this manner, utilizing accurate cow scoring, penning, and moving between pens, the feed formulations for the diets can be corrected to address any variations caused by ingredient variability, while maximizing herd productivity and health.

Under the present invention, the nutritionist can designate or receive from a third party service designations for these cow regroupings for early/mid lactation vs. late lactation cows. These pen allocations can be updated automatically on a daily, weekly or other time basis.

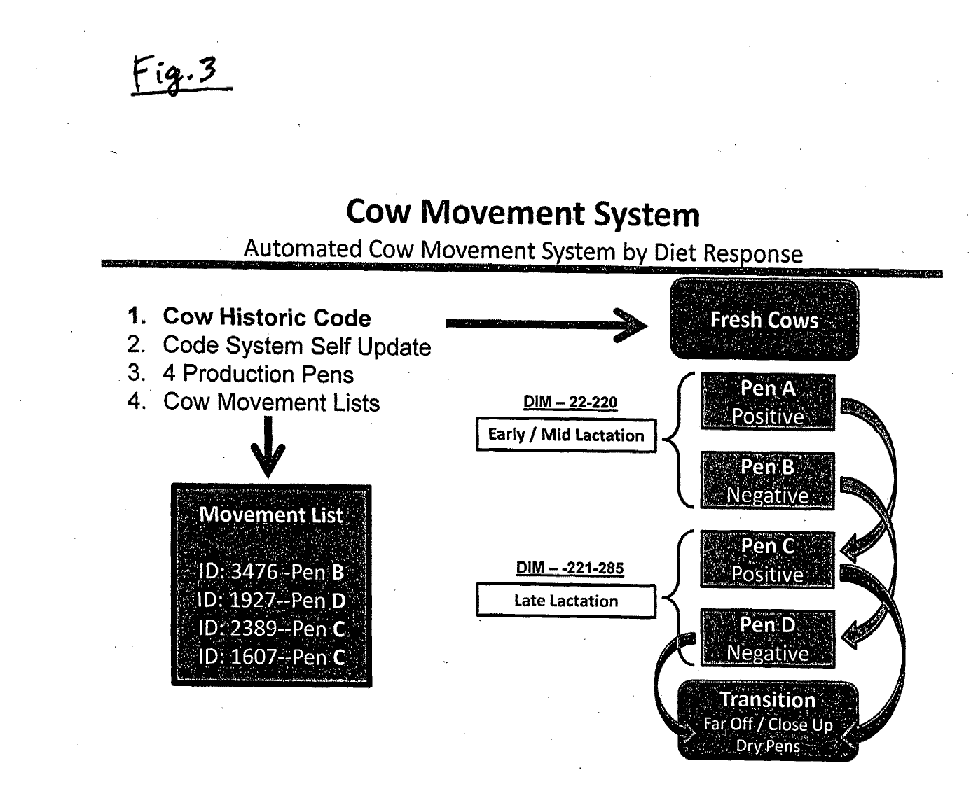

Finally, the cows will in due course need to be moved from the early/mid lactation phase pens A and B to the late lactation phase pens C and D. As shown in FIG. 3, normally the positive-response early/mid lactation cows in Pen A would be automatically moved to the Pen C for positive-response late lactation cows. For purposes of this invention, the “early/mid lactation” stage covers approximately 22-220 days in milk (“DIM”), while “late lactation” stage covers approximately 221-285 DIM. However, not all the cows in Pen A may be ready to move to the corresponding late lactation phase positive response Pen C at the same time. For example, if a cow is exhibiting exemplary milk production and stability while in early/mid lactation phase Pen A, then it may make sense to leave her in Pen A beyond the normal Day 221 onset for the late lactation phase. Once her milk productivity and stability numbers start to decline, then it becomes time to move her to Pen C to receive a lower-fermentation diet. Therefore, cows should be moved in accordance with the principles of this invention between the early/mid lactation-stage and late lactation-stage pens based upon their economic output instead of the passage of the calendar, as is customary in the dairy industry. Of course, the cows should also be constantly scored for their relative fermentability, productivity, and stability to determine whether they should be moved between the positive and negative-response pens for their production stage.

In this manner, the cows are evaluated individually for productivity response and animal health. They are penned accordingly and fed targeted feed rations for their productivity and health status. Using the principles of this invention incorporating determination in real-time of the fiber and starch digestibility, compositional characteristics, and total ration fermentation value for a feed ration, the overall productivity and health of the dairy cow herd can be maximized.

The present invention also includes using traditional real-time screening techniques, such as wet chemistry, to determine the starch and/or fiber digestibility characteristics of a particular crop in the field or a crop that is stored on an identity preserved basis. The invention, therefore includes, analyzing the starch, fiber, and/or compositional digestibility of an identity preserved crop in real-time, using techniques described herein or other techniques known in the art, and using that information to prepare feed formulations that optimize ruminant productivity.

The present invention also includes growing a crop at a particular location and determining the starch degradability characteristics of the crop plant used as grain or NDF digestibility if used as a forage in real time, before or after harvest, by NIRS. The crop plant or plant parts are stored on an identity preserved basis. Based on specific diet requirements, conservation methods such as high-moisture fermentation or harvesting field dried, and processing including either rolling or grinding, and steam flaking are used to alter measured starch degradability. Once a specific starch degradability target/requirement for a ruminant herd is determined, a blending process of mixing fast and slow starch degradation properties that have been accurately measured according to the present invention are incorporated into a feed formulation for optimum ruminant productivity.

It is understood that the present invention is applicable to corn, alfalfa, and other forage crops, and can also be used to characterize forage sources in real time. Thus, the term “crop plant” or “crop” is meant to include any plant that is used as silage, grain or other plant based feed ingredient for ruminant animals.

The plant characteristics, energy (digestibility), protein and fiber content of both corn grain and corn forage is affected by the interaction of genetics by environment (G×E). Thus, according to the present invention, real-time characterization of each source of starch (grain) and NDF (fiber) is necessary to accurately formulate diets for ruminates. Once an animal production target is determined, a total mixed ration (TMR) is designed by combining energy, protein, fiber, vitamins and mineral ingredients into a mixer wagon based on predetermined metabolizable energy (ME) targets, crude protein and meeting adequate and sufficient fiber requirements.

Meeting the total ration NDF target and the level of NDF as a percentage of the total forage in the diet determines the forage component of the base diet. An adjusted ME value for the forage sources is determined to account for the energy contribution (NDF digestibility) from the forage NDF.

The production requirement of the diet and the forage/fiber composition of the diet will determine the optimal amount and source of supplemental starch, with either a fast, slow or mid-point of starch degradability needed to make the most feed efficient, productive and healthy diet formulation. The forage characteristics of the diet also determines the optimum moisture content of the starch, either dry grain (15.5%) or high moisture grain, such as high moisture corn (HMC) at 28-32% by weight, and which conservation and processing methods are advantageous to the production and health impact of the diet.

It is understood, therefore, that the present invention is a system that optimizes a ruminant feed formulation by analysis of identity preserved feed components on a real-time basis. It is further understood that the present invention includes using various methods of measuring, in real time, crop plant characteristics.

The above specification, drawings, and data provide a complete description of the feeding method and resulting feed compositions of the present invention. Since many embodiments of the invention can be made without departing from the spirit and scope of the invention, the invention resides in the claims hereinafter appended.

What is claimed is:

1. A system for characterizing in real time crop plants to be used in a feed ration to optimize productivity of a ruminant animal that consumes such feed ration, such system comprising:
(a) determination of starch digestibility characteristics of a set of crop plant samples comprising grain of said crop plant samples;
(b) development of a prediction equation based on said starch digestibility characteristics;
(c) obtaining a grain sample from a crop plant;
(d) determination in real time of the starch digestibility characteristics by NIRS of said sample by inputting electronically recorded near infrared spectrum data from said NIRS into the equation;
(e) storing and/or milling said grain on an identity preserved basis; and
(f) determination of the amount of such crop plant to incorporate into a feed ration based upon the starch digestibility characteristics determined in step (d).

2. The real-time characterization system according to claim 1, wherein the crop plant is brown midrib corn.

3. The real-time characterization system according to claim 1, wherein the crop plant is dual-purpose corn.

4. The real-time characterization system according to claim 1, wherein the crop plant is leafy corn.

5. The real-time characterization system according to claim 1, wherein the crop plant is alfalfa.

6. The real-time characterization system according to claim 1, wherein the crop plant is grass.

7. The real-time characterization system according to claim 1 further comprising prediction of starch digestibility characteristics of the crop plant samples comprising grain of said crop plant samples at various particle sizes, based upon the prediction equations.

8. A system for characterizing in real time crop plants to be used in a feed ration to optimize productivity of a ruminant animal that consumes such feed ration, such system comprising:
(a) determination of starch digestibility characteristics of grain from genetically different crop plants;
(b) determination of dNDF characteristics of genetically different crop plants for use as forage;
(c) development of prediction equations based on said starch digestibility and dNDF characteristics;
(d) obtaining grain samples for use as feed supplements and crop plants for use as forage;
(e) determination of starch and NDF digestibility characteristics by NIRS of said grain samples and said crop plants by inputting electronically recorded near infrared spectrum data relating to said characteristics into said equations; and
(f) determination the amounts of said grain and said crop plants to incorporate into a feed formulation based on the starch and NDF digestibility characteristics determined in step (e).

9. The real-time characterization system according to claim 8, wherein the crop plant is brown midrib corn.

10. The real-time characterization system according to claim 8, wherein the crop plant is dual-purpose corn.

11. The real-time characterization system according to claim 8, wherein the crop plant is leafy corn.

12. The real-time characterization system according to claim 8, wherein the crop plant is alfalfa.

13. The real-time characterization system according to claim 8, wherein the crop plant is grass.

14. The real-time characterization system according to claim 1 further comprising prediction of starch digestibility characteristics of the crop plant samples comprising grain of said crop plant samples at various particle sizes, based upon the prediction equations.

15. The real-time characterization system according to claim 1 further comprising prediction of forage digestibility characteristics of the crop plant samples comprising forage of said crop plant samples at various particle sizes, based upon the prediction equations.

16. The real-time characterization system according to claim 1, wherein such system comprises a computer-based tool incorporating such prediction equations.

17. The real-time characterization system according to claim 1, wherein such system is portable.

18. The real-time characterization system according to claim 1 further comprising calculation of a total ration fermentation value for the resulting feed ration based upon the characterized values of the crop plants to determine the total level of fermentability delivered to the ruminant animal that consumes the feed ration.

19. The real-time characterization system according to claim 1 further comprising calculation of a flash fermentation value for the resulting feed ration based upon the characterized values of the crop plants to predict the total level of fermentability that will be delivered to the rumen of the ruminant animal that consumes the feed ration within an initial residence period after consumption.

20. The real-time characterization system according to claim 19, wherein the initial residence period is about two hours.
